(19) United States
(12) Patent Application Publication
(10) Pub. No.: US 2024/0058302 A1
(43) Pub. Date: Feb. 22, 2024

(54) METHOD OF INDUCTION OF TUMOR ASSOCIATED ANTIGENS WITH BRYOSTATIN

(71) Applicant: BryoLogyx Inc., (US)

(72) Inventors: Stephen Becker, Danville, CA (US); Sathapana Kongsamut, Danville, CA (US)

(21) Appl. No.: 17/766,725

(22) PCT Filed: Oct. 8, 2020
(86) PCT No.: PCT/US2020/054693

§ 371 Date: Apr. 5, 2022

Related U.S. Application Data
(60) Provisional application No. 62/913,605, filed on Oct. 10, 2019.

(51) Int. Cl. A61K 31/365; C07K 16/28; A61K 45/06; A61P 35/00

(52) U.S. Cl. CPC A61K 31/365; C07K 16/2803; A61K 45/06; A61P 35/00; A61K 2039/505

(57) ABSTRACT

Bryostatin-1 stimulates the expression of tumor associated antigens in hematologic malignancies, enabling tumors to be recognized by immune therapies, thus increasing the clinical responses and duration of those responses to various immune therapies. Accordingly, methods of treatment based on this observation are provided herein.

### CROSS-REFERENCE TO RELATED APPLICATIONS

This application claims benefit of priority under 35 U.S.C. § 119(e) of U.S. Ser. No. 62/913,605, filed Oct. 10, 2019, the entire contents of which is incorporated herein by reference in its entirety.

### BACKGROUND OF THE INVENTION

### Field of the Invention

The invention relates generally to cancer and more specifically to induction of tumor associated antigens for improved immune surveillance of aberrant cells in a subject.

### Background Information

Cancer is a disease where cells escape normal regulatory processes, transform and grow rapidly. These rapidly growing cells may arise in blood (hematological malignancy), or as solid tumors in various organs. Abnormal, rapidly growing cells are distinguished by the expression of unique antigens that are recognized by the host immune system and are held in check by various immune mechanisms. When transformed cells escape normal immune regulatory mechanisms, cells that grow rapidly become malignant tumors, either in blood or as solid tumors.

Such tumors express various antigens termed tumor associated antigens (TAA), cellular proteins constitutively expressed by tumor cells. These antigens provide a stimulus to host immune effector function and in addition serve as targets for immunotherapies, notably monoclonal antibodies, antibody-drug conjugates, chimeric antigen receptor (CAR)-T cells and CAR-natural killer (NK) cells, are examples of such immuno-oncology approaches used in various malignancies.

An important aspect of these immune-mediated therapies is the adequate expression of tumor antigens that enable the immunotherapies to recognize aberrant cells for destruction. In the B-cell malignancies, TAAs may be specific for tumor types, or may be shared among tumors given their primary origin in B-cells. CD19 and CD20, among other TAAs are associated with non-Hodgkin lymphomas, and are the targets of several approved oncology agents. Similarly, other TAAs are associated with acute lymphoblastic leukemia (CD22), acute myeloid leukemia (CD33 and CD123) and multiple myeloma (CD38 and B-cell maturation antigen, or BCMA), by way of example. Several studies across these hematologic malignancies have demonstrated that the quantitative expression, or density, of TAA is an important factor in the efficacy of immunotherapies. Although less common, T-cell malignancies, such as T-cell acute lymphoblastic leukemia, also express a variety of tumor-associated antigens.

Bryostatin-1 and its analogs are modulators of protein kinase C (PKC), stimulating PKC activity at low concentrations and causing downregulation of PKC at high concentrations. Stimulation of PKC induces the expression of certain genes, including those of the tumor associated antigens, and interaction with various intracellular signaling mechanisms. For example, bryostatin-1 has been shown to increase the cell-surface expression of CD22, a tumor associated antigen in B-cell tumors such as acute and chronic lymphoblastic and lymphocytic leukemia.

### SUMMARY OF THE INVENTION

The invention is based on the finding that bryostatin-1 and analogs thereof can induce the expression of tumor associated antigens (TAAs) resulting in improved recognition of aberrant (e.g. cancer) cells expressing these antigens, and the improved ability of immune therapies to target and kill these aberrant cells.

In one embodiment the present invention provides a method of treating cancer in a subject in need thereof by administering to the subject bryostatin-1 or a functional analog thereof and an immunotherapeutic agent. In one aspect, the cancer is a hematologic cancer such as a B-cell lymphoma, a T-cell malignancy, non-Hodgkin's lymphoma (NHL), mantle cell lymphoma (MCL), multiple myeloma (MINI), acute myelogenous leukemia (AML), chronic myelogenous leukemia (CIVIL), myelodysplastic syndrome, acute lymphoblastic leukemia (ALL), chronic lymphocytic leukemia (CLL), and hairy cell leukemia (HCL). In certain aspects, the immunotherapeutic agent is a monoclonal antibody (Mab), a bispecific antibody, an antibody-drug conjugate (ADC), a chimeric antigen receptor (CAR)-T cell, a chimeric antigen receptor natural killer (CAR-NK) cell, an anti-tumor vaccine or a combination thereof. In one aspect, the immunotherapeutic agent is an anti-CD22 antibody, for example inotuzumab. In certain aspects, the immunotherapeutic agent is a CAR-T cell or a CAR-NK targeting CD22. In one aspect, the ADC is inotuzumab ozogamicin. In various aspects, the subject is additionally administered an agent such as an antibody directed against a TAA, a chemotherapeutic agent, an ADC, a vaccine, an immunomodulatory drug, an immune metabolism modifying drug, a targeted therapy, radiation, an anti-angiogenesis agent, CAR-T therapy, CAR-NK therapy, an agent that reduces immune-suppression, or a combination thereof. In one aspect, the immunotherapeutic agent is administered prior to, simultaneously with or following administration of bryostatin-1 or a functional analog thereof.

In one aspect, the immunotherapeutic agent binds to or targets a protein selected from CD19, CD20, CD22, CD33, CD37, CD38, CD123 and BCMA. In an additional aspect, there is an increase in CD5, CD19, CD20, CD22, CD33, CD38, CD123, and/or BCMA expression following administration of bryostatin-1 or a functional analog thereof and the immunotherapeutic agent. In certain aspects, the subject has CLL and there is an increase in CD19 and/or CD22 expression following administration of bryostatin-1 or a functional analog thereof and the immunotherapeutic agent. In various aspects, the subject has multiple myeloma and there is an increase in CD 38 and/or BCMA expression following administration of bryostatin-1 or a functional analog thereof and the immunotherapeutic agent.

In an additional embodiment, the present invention provides a method of initiating, enhancing, or prolonging an anti-tumor response in a subject in need thereof by administering to the subject bryostatin-1 or a functional analog thereof, and an immunotherapeutic agent. In one aspect, the subject has a hematological cancer selected from a B-cell lymphoma, a T-cell malignancy, non-Hodgkin's lymphoma (NHL), mantle cell lymphoma (MCL), multiple myeloma (MM), acute myelogenous leukemia (AML), chronic myelogenous leukemia (CML), myelodysplastic syndrome, acute lymphoblastic leukemia (ALL), chronic lymphocytic leukemia (CLL), and hairy cell leukemia (HCL). In a further aspect, the immunotherapeutic agent is a monoclonal antibody (Mab), a bispecific antibody, an antibody-drug conjugate (ADC), a chimeric antigen receptor T (CAR-T) cell, chimeric antigen natural killer (CAR-NK) cell, an anti-tumor vaccine or a combination thereof. In one aspect, immunotherapeutic agent is administered prior to, simultaneously with or following administration of bryostatin-1 or a functional analog thereof. In certain aspects, the initiating, enhancing or prolonging an anti-tumor response is an increase in CD5, CD19, CD20, CD22, CD33, CD38, CD123, and/or BCMA expression following administration of bryostatin-1 or a functional analog thereof and the immunotherapeutic agent.

In a further embodiment, the present invention provides a method of treating a cancer resistant to an immunotherapeutic agent in a subject by administering bryostatin-1 or a functional analog thereof and the immunotherapeutic agent to the subject. In one aspect, the cancer is a hematological cancer such as B-cell lymphomas, T-cell malignancies, non-Hodgkin's lymphoma (NHL), mantle cell lymphoma (MCL), multiple myeloma (MM), acute myelogenous leukemia (AML), chronic myelogenous leukemia (CIVIL), myelodysplastic syndrome, acute lymphoblastic leukemia (ALL), chronic lymphocytic leukemia (CLL) and hairy cell leukemia (HCL). In an additional aspect, the immunotherapeutic agent is a monoclonal antibody, a bispecific antibody, an antibody-drug conjugate, a chimeric antigen receptor (CAR)-T cell, chimeric antigen receptor natural killer (CAR-NK) cell, an anti-tumor vaccine or a combination thereof. In certain aspects, the immunotherapeutic agent binds to or targets a protein selected from CD19, CD20, CD22, CD33, CD37, CD38, CD123 and BCMA. In various aspects, there is an increase in CD5, CD19, CD20, CD22, CD33, CD38, CD123, and/or BCMA expression following administration of bryostatin-1 or a functional analog thereof and the immunotherapeutic agent.

### BRIEF DESCRIPTION OF THE DRAWINGS

FIGS. 1A-1C shows representative concentration response curves showing bryostatin-1 upregulates cell surface antigens in REH (ALL), AML193 (AML) and NCI-H929 (MM) cells. FIG. 1A: CD22, FIG. 1B: CD123, and FIG. 1C: CD269 (BCMA).

FIG. 2 shows that CD5 expression increases in response to bryostatin-1 after 24 h exposure in MM patients but not in healthy donors or CLL patients.

FIG. 3 shows that CD19 expression in response to bryostatin-1 after 72 h exposure, showing increases in healthy donors and CLL patients but not MM patients.

FIG. 4 shows the response to CD22-directed CAR-T and bryostatin-1 in a PDX model using tumor cells derived from relapsed, low-expressing TAA patients.

### DETAILED DESCRIPTION OF THE INVENTION

The invention is based on the finding that bryostatin-1 and functional analogs thereof can induce the expression of TAA resulting in improved recognition of aberrant (e.g., cancer) cells expressing these antigens and the improved ability of immune therapies to target and kill these aberrant cells.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

Bryostatin-1 induces the expression of tumor associated antigens (TAAs) in B-cell tumors (hematologic malignancies), enabling these tumors to be recognized by immune therapies, and increase the clinical responses to various immune therapies. Analogs of bryostatin-1 stimulate the expression of TAAs in hematologic malignancies, enabling these tumors to be recognized by the immune system and immune therapies, and increase the clinical responses to various immune therapies. Bryostatin-1 and analogs induce the expression of TAAs in leukemias, lymphomas and myelomas, specifically in myelogenous leukemias, non-Hodgkin lymphomas and multiple myeloma, enabling these tumors to be recognized by immune therapies, and increase the clinical responses to various immune therapies.

A major impediment to the efficacy of cancer immunotherapy relates to the scarcity of TAAs that function to engage host immune function, and act as cellular targets for immunotherapeutic agents including monoclonal antibodies, antibody-drug conjugates, CAR-T and CAR-NK agents. Cell membrane expression of TAAs, also referred to as TAA density has been described as a predictor of response to immunotherapeutic agents. Indeed, the clinical failures, or relapses, that occur following CD19 CAR-T in acute lymphoblastic leukemia is associated with the loss, and cellular internalization of the target CD19 molecule.

In one embodiment the present invention provides a method of treating cancer in a subject in need thereof by administering to the subject bryostatin-1 or a functional analog thereof and an immunotherapeutic agent. In one aspect, the cancer is a hematologic cancer such as B-cell lymphomas, T-cell malignancies, non-Hodgkin's lymphoma (NHL), mantle cell lymphoma (MCL), multiple myeloma (MM), acute myelogenous leukemia (AML), chronic myelogenous leukemia (CIVIL), myelodysplastic syndrome, acute lymphoblastic leukemia (ALL), chronic lymphocytic leukemia (CLL) and hairy cell leukemia (HCL). In certain aspects, the immunotherapeutic agent is a monoclonal antibody, a bispecific antibody, an antibody-drug conjugate (ADC), a chimeric antigen receptor (CAR)-T cell, a chimeric antigen receptor natural killer (CAR-NK) cell, an anti-tumor vaccine or a combination thereof. In an aspect, the immunotherapeutic agent is an anti-CD22 antibody. In certain aspect, the immunotherapeutic agent is a CAR-T cell or a CAR-NK cell targeting CD22. In one aspect, ADC is inotuzumab ozogamicin. In various aspects, the subject is additionally administered an agent such as an antibody directed against a (TAA), a chemotherapeutic agent, an ADC, a vaccine, an immunomodulatory drug, an immune metabolism modifying drug, a targeted therapy, radiation, an anti-angiogenesis agent, CAR-T therapy, CAR-NK therapy, an agent that reduces immune-suppression, or a combination thereof. In one aspect, the immunotherapeutic agent is administered prior to, simultaneously with or following administration of bryostatin-1 or a functional analog thereof.

In one aspect, the immunotherapeutic agent binds to or targets a protein selected from CD19, CD20, CD22, CD33, CD37, CD38, CD123 and BCMA. In an additional aspect, there is an increase in CD5, CD19, CD20, CD22, CD33, CD38, CD123, and/or BCMA expression following administration of bryostatin-1 or a functional analog thereof and the immunotherapeutic agent. In certain aspects, the subject has CLL and there is an increase in CD19 and CD22 expression following administration of bryostatin-1 or a functional analog thereof and the immunotherapeutic agent. In various aspects, the subject has multiple myeloma and there is an increase in CD38 and/or BCMA expression following administration of bryostatin-1 or a functional analog thereof and the immunotherapeutic agent.

Bryostatin-1 or functional analogs (also known by some as “bryologs”) thereof refer to a group of macrolide lactones known to be potent modulators of protein kinase C (PKC). Bryostatin-1 is derived from a marine bryozoan, Bugula neritina, and is a potent modulator of the protein kinase C (PKC) enzyme system, a family of proteins involved in cellular signaling, cell proliferation and cell death. As such bryostatin-1 has application across several therapeutic indications. To date, 20 different bryostatins have been isolated. Bryostatin-1, is the best characterized, and the only member of the bryostatin family to have been evaluated in human clinical trials. At low concentrations, it is a potent PKC activator, and results in cell activation, signaling and induction of transcription, and the cell membrane expression of tumor and/or human immunodeficiency virus (HIV) antigens. Bryostatin-1 thus makes these cells more visible to the host immune system, and to exogenously administered immunotherapeutic agents that target these antigens. Examples Bryostatin-1 and bryostatin analogs are known in the art and can be found for example in U.S. Pat. No. 8,816,122, herein incorporated by reference.

As used hereon, the term “functional analogs” refer to chemical compounds that have similar physical, chemical, biochemical, or pharmacological properties. Functional analogs are not necessarily structural analogs with a similar chemical structure.

The term “cancer” refers to a group diseases characterized by abnormal and uncontrolled cell proliferation starting at one site (primary site) with the potential to invade and to spread to others sites (secondary sites, metastases) which differentiate cancer (malignant tumor) from benign tumor. Virtually all the organs can be affected, leading to more than 100 types of cancer that can affect humans. Cancers can result from many causes including genetic predisposition, viral infection, exposure to ionizing radiation, exposure to environmental pollutant, tobacco and or alcohol use, obesity, poor diet, lack of physical activity or any combination thereof. As used herein, “neoplasm” or “tumor” including grammatical variations thereof, means new and abnormal growth of tissue, which may be benign or cancerous. In a related aspect, the neoplasm is indicative of a neoplastic disease or disorder, including but not limited, to various cancers. For example, such cancers can include prostate, pancreatic, biliary, colon, rectal, liver, kidney, lung, testicular, breast, ovarian, pancreatic, brain, and head and neck cancers, melanoma, sarcoma, multiple myeloma, leukemia, lymphoma, and the like.

Cancer that begins in blood-forming tissue, such as the bone marrow, or in the cells of the immune system are referred to as hematologic cancer, or blood cancer. Hematologic cancers affect the production and function of blood cells, and are classified in three main types: leukemia, lymphoma, and multiple myeloma.

As used herein, “leukemia” refers to a blood cancer caused by the rapid production of abnormal white blood cells. Examples of leukemia include acute lymphoblastic leukemia (ALL), acute myeloid leukemia (AML), chronic lymphocytic leukemia (CLL), chronic myelogenous leukemia (CML), and hairy cell leukemia.

As used herein, “lymphoma” refers to a type of blood cancer that affects the lymphatic system. Examples of lymphoma include AIDS-related lymphoma, cutaneous T-cell lymphoma, Hodgkin's lymphoma, mycosis fungoides, non-Hodgkin's lymphoma, mantle cell lymphoma, primary central nervous system lymphoma, Sézary syndrome, cutaneous T-Cell lymphoma, and Waldenström macroglobulinemia.

As used herein, “myeloma” is a cancer of the plasma cells. Examples of myeloma include chronic myeloproliferative neoplasms, Langerhans cell histiocytosis, multiple myeloma, plasma cell neoplasm, myelodysplastic syndromes, and myelodysplastic/myeloproliferative neoplasms.

As used herein, the term “immunotherapeutic agent” refers to a therapeutic agent that elicits an immune response. Examples of an immunotherapeutic agent include a monoclonal antibody (Mab), a bispecific antibody, an antibody-drug conjugate (ADC), a chimeric antigen receptor T cell (CAR-T), a chimeric antigen receptor natural killer (CAR-NK) cell, an anti-tumor vaccine or a combination thereof.

The term “antibody,” as used herein, refers to immunoglobulin molecules and immunologically active portions of immunoglobulin molecules, i.e., molecules that contain an antigen binding site that immunospecifically binds an antigen. “Native antibodies” and “intact immunoglobulins”, or the like, are usually heterotetrameric glycoproteins of about 150,000 daltons, composed of two identical light (L) chains and two identical heavy (H) chains. The light chains from any vertebrate species can be assigned to one of two clearly distinct types, called kappa (x) and lambda (k), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of immunoglobulins are called α, δ, ε, γ, and μ, respectively. The subunit structures and three-dimensional configurations of different classes of immunoglobulins are well known.

Experimentally, antibodies can be cleaved with the proteolytic enzyme papain, which causes each of the heavy chains to break, producing three separate antibody fragments. The two units that consist of a light chain and a fragment of the heavy chain approximately equal in mass to the light chain are called the Fab fragments (i.e., the “antigen binding” fragments). The third unit, consisting of two equal segments of the heavy chain, is called the Fc fragment. The Fc fragment is typically not involved in antigen-antibody binding, but is important in later processes involved in ridding the body of the antigen. “Fv” is the minimum antibody fragment which contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. “Single-chain Fv” or “sFv” antibody fragments comprise the VH and VL domains of antibody, wherein these domains are present in a single polypeptide chain. Preferably, the Fv polypeptide further comprises a polypeptide linker between the VH and VL domains which enables the sFv to form the desired structure for antigen binding. For a review of sFv see Pluckthun in The Pharmacology of Monoclonal Antibodies, vol. 113, Rosenburg and Moore eds., Springer-Verlag, New York, pp. 269-315 (1994).

A bispecific monoclonal antibody (BsMAb, BsAb) is an artificial protein that can simultaneously bind to two different types of antigen. IgG like bispecific antibodies have a traditional monoclonal antibody (mAb) structure of two Fab arms and one Fc region, except the two Fab sites bind different antigens. The most common types are called trifunctional antibodies, as they have three unique binding sites on the antibody: the two Fab regions, and the Fc region. Each heavy and light chain pair is from a unique mAb. The Fc region made from the two heavy chains forms the third binding site. There are other bsMabs that lack an Fc region entirely. These include chemically linked Fabs, consisting of only the Fab regions, and various types of bivalent and trivalent single-chain variable fragments (scFvs). There are also fusion proteins mimicking the variable domains of two antibodies.

There are a number of immuno-oncology drugs and therapies that can be used in combination with bryostatin-1 to treat cancer in patients. Examples of antibodies and ADCs for the treatment of cancer whose efficacy may be improved by combination with bryostatin-1 and analogs thereof include but are not limited to Blycyto (blinatumomab), Bexxar (tositumomab), Rituxan/MabThera (Rituximab), Zevalin (ibritumomab tiuxetan), Arzerra (ofatumumab), Gazyva (obinutuzumab), Besponsa (inotuzumab ozogamycin), Lumoxiti (moxetumomab pasudotox-tdfk), Adcetris (brentuximab vedotin), Mylotarg (gemtuzumab oxogamicin), Darzalex (daratumumab), Raptiva (efalizumab), Yervoy (ipilimumab), Opdivo (nivolumab), Keytruda (pembrolizumab), Kymriah (tisagenlecleucel), Yescarta (axicabtagene ciloleucel), Imfinzi (durvalumab), Tecentriq (atezolizumab), Bavencio (avelumab), and Libtayo (cemiplimab). Additionally, the efficacy of following antibodies and drugs may be improved by bryostatin-1 and its analogs: Avastin (bevacizumab), Cimzia (certolizumab pegol, CDP 870), Prolia/Xgeva (denosumab), Erbitux (cetuximab), Herceptin (trastuzumab), Humira (adalimumab), Portazza (necitumumab, IMC-IIF 8), LeukoScan (sulesomab), MabCampath (alemtuzumab), Tysabri (natalizumab), Vectibix (panitumamab), Remicade (infliximab), ReoPro (abciximab), Simulect (basiliximab), Synagis (palivizumab), Actembra (tocilizumab), Gilotrif (afatinib), Lynparza (olaparib), Perjeta (pertuzumab), Bosulif (bosutinib), Cabometyx (cabozantinib), trastuzumab-dkst (Ogivri), Sutent (sunitinib malate), Alecensa (alectinib), Calquence (acalabrutinib), Imbruvica (ibrutinib), Verzenio (abemaciclib), Aliqopa (copanlisib), Nerlynx (neratinib), Tarceva (erlotinib).

Antibody-drug conjugates or ADCs are a class of biopharmaceutical drugs designed as a targeted therapy for treating cancer. ADCs are complex molecules composed of an antibody linked to a biologically active cytotoxic (anticancer) payload or drug. Antibody-drug conjugates are examples of bioconjugates and immunoconjugates. ADCs combine the targeting capabilities of monoclonal antibodies with the cancer killing ability of cytotoxic drugs. An anticancer drug is coupled to an antibody that specifically targets a certain tumor antigen. Antibodies attach to the antigens on the surface of cancer cells. The biochemical reaction between the antibody and the target protein (antigen) triggers a signal in the tumor cells, which then absorbs or internalizes the antibody together with the linked cytotoxin. After the ADC is internalized, the cytotoxin kills the cancer cell. This targeting, limits side effects and gives a wider therapeutic window than other chemotherapeutic agents. Examples of antibody-drug conjugates include Gemtuzumab ozogamicin, Brentuximab vedotin, Trastuzumab emtansine, Inotuzumab ozogamicin, Polatuzumab vedotin-piiq, Enfortumab vedotin, Trastuzumab deruxtecan, Sacituzumab govitecan and Belantamab mafodotin.

The term “CAR-transduced T cells” and the term “CAR-T cells” are used interchangeably herein and refer to T cells that have been genetically modified to express chimeric antigen receptors (CARs) targeting a specific antigen. According to particular aspects, the antigen presents on the surface of cancer cells. According to some aspects, the cancer cells are hematological cancer cells. According to some aspects, the CAR-T cells express CARs targeting an antigen selected from but not limited to CD19, CD20, CD22, CD30, CD33, CD38, CD123, FLT3 and BCMA.

CAR-T cells are isolated from a subject and undergo ex vivo genetic manipulation using either lentiviral or retroviral vectors or non-viral gene transfer systems, to express the engineered CARs specific for particular tumor targets. These reprogrammed CAR-T cells are then expanded, selected if necessary, and infused into the subject after they have received an immunosuppressive preparative regimen. In some aspects, the ex vivo genetic manipulation of the isolated T cells can be performed before or after the process of mitochondrial enrichment.

After transplantation, the CAR-T cells undergo antigen engagement and amplify in the peripheral blood, from where they travel to tumor sites and identify and kill tumor cells expressing the corresponding antigen. This can trigger extensive proliferation of CAR-T cells and the release of tumor antigens, which activate the subject's immune system to recruit non-CAR-T immune cells, thus eliciting further antitumor responses in a process known as cross-priming from epitope spreading.

The term CAR-NK refers to natural killer (NK) cells that have been engineered to express chimeric antigen receptors (CARs) targeting a specific antigen. NK cells are part of the innate immune system. Like CAR-T cells, CAR-NK cells will destroy the cell possessing the antigen that is the target of the CAR.

The term “vaccine” relates to a pharmaceutical preparation (pharmaceutical composition) or product that upon administration induces an immune response, in particular a cellular immune response, which recognizes and attacks a pathogen or a diseased cell such as a cancer cell. A vaccine may be used for the prevention or treatment of a disease. The term “individualized cancer vaccine” concerns a particular cancer patient and means that a cancer vaccine is adapted to the needs or special circumstances of an individual cancer patient.

As used herein, the term “vaccination” relates to the administration of a vaccine, a pharmaceutical preparation (pharmaceutical composition) or product that upon administration induces an immune response, in particular a cellular immune response, which can recognize and attack a pathogen or a diseased cell such as a cancer cell. A vaccine may be used for the prevention or treatment of a disease. The term “immune response” refers to an integrated bodily response to an antigen and preferably refers to a cellular immune response or a cellular as well as a humoral immune response. The immune response may be protective/preventive/prophylactic and/or therapeutic. Inducing an immune response” may mean that there was no immune response against a particular antigen before induction, but it may also mean that there was a certain level of immune response against a particular antigen before induction and after induction said immune response is enhanced. Thus, “inducing an immune response” also includes “enhancing an immune response”. Preferably, after inducing an immune response in a subject, said subject is protected from developing a disease such as a cancer disease or the disease condition is ameliorated by inducing an immune response. For example, an immune response against a tumor expressed antigen may be induced in a donor, which will help the recipient subject having a cancer disease to fight against the cancer disease. Inducing an immune response in this case may mean that the disease condition of the subject is ameliorated, that the subject does not develop metastases, or that the subject being at risk of developing a cancer disease does not develop a cancer disease.

An “antigen” or “tumor-associated antigen” refers to any substance that will elicit an immune response, such as any tumor-associated substance. In particular, an “antigen” relates to any substance, preferably a peptide or protein, that reacts specifically with antibodies or T-lymphocytes (T cells). According to the present invention, the term “antigen” comprises any molecule which comprises at least one epitope. Preferably, an antigen in the context of the present invention is a molecule which, optionally after processing, induces an immune reaction. According to the present invention, any suitable antigen may be used, which is a candidate for an immune reaction, wherein the immune reaction is preferably a cellular immune reaction. In the context of the embodiments of the present invention, the antigen is preferably presented by a cell, preferably by an antigen presenting cell which includes a diseased cell, in particular a cancer cell, in the context of MEW molecules, which results in an immune reaction against the antigen. An antigen is preferably a product which corresponds to or is derived from a naturally occurring antigen. Such naturally occurring antigens include tumor antigens.

As used herein the terms “tumor associated antigen” and “TAA” refer to proteins expressed on the surface of tumor cells that can be recognized by the immune system and immunotherapy agents. These proteins trigger an immune response. The quantitative expression of the tumor associated antigen (also referred to as the tumor antigen density) is a known correlate of clinical response. These antigens may be expressed in normal cells as well, reflecting their constitutive expression and normal biologic function. Examples of TAA included CD5, CD19. CD20, CD33, CD37, CD38, CD123 and B-cell maturation antigen (BCMA).

The term “treatment” is used interchangeably herein with the term “therapeutic method” and refers to both 1) therapeutic treatments or measures that cure, slow down, lessen symptoms of, and/or halt progression of a diagnosed pathologic conditions or disorder, and 2) and prophylactic/preventative measures. Those in need of treatment may include individuals already having a particular medical disorder as well as those who may ultimately acquire the disorder (e.g., those needing preventive measures).

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological. The effective amount can be determined as described herein.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration. The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectables, implantable sustained-release formulations, lipid complexes, etc.

In some aspects administration can be in combination with one or more additional therapeutic agents. The phrases “combination therapy”, “combined with” and the like refer to the use of more than one medication or treatment simultaneously to increase the response. The composition of the present invention might for example be used in combination with other drugs or treatment in use to treat cancer. Specifically, the administration of the composition of the present invention to a subject can be in combination with any anti-cancer therapies. Such therapies can be administered prior to, simultaneously with, or following administration of the composition of the present invention.

In certain aspects, the subject is additionally administered an agent such as an antibody against a TAA, a chemotherapeutic agent, an ADC, a vaccine, an immunomodulatory drug, an immune metabolism modifying agent, a targeted therapy, radiation, an anti-angiogenesis agent, CAR-T therapy, CAR-NK therapy, an agent the reduces immune-suppression or a combination thereof.

The term “anti-cancer therapy” or “anti-cancer treatment” as used herein is meant to refer to any treatment that can be used to treat cancer, such as surgery, radiotherapy, chemotherapy, immunotherapy, and checkpoint inhibitor therapy.

Examples of chemotherapy include treatment with a chemotherapeutic, cytotoxic or antineoplastic agents including, but not limited to, (i) anti-microtubules agents comprising vinca alkaloids (vinblastine, vincristine, vinflunine, vindesine, and vinorelbine), taxanes (cabazitaxel, docetaxel, larotaxel, ortataxel, paclitaxel, and tesetaxel), epothilones (ixabepilone), and podophyllotoxin (etoposide and teniposide); (ii) antimetabolite agents comprising anti-folates (aminopterin, methotrexate, pemetrexed, pralatrexate, and raltitrexed), and deoxynucleoside analogues (azacitidine, capecitabine, carmofur, cladribine, clofarabine, cytarabine, decitabine, doxifluridine, floxuridine, fludarabine, fluorouracil, gemcitabine, hydroxycarbamide, mercaptopurine, nelarabine, pentostatin, tegafur, and thioguanine); (iii) topoisomerase inhibitors comprising Topoisomerase I inhibitors (belotecan, camptothecin, cositecan, gimatecan, exatecan, irinotecan, lurtotecan, silatecan, topotecan, and rubitecan) and Topoisomerase II inhibitors (aclarubicin, amrubicin, daunorubicin, doxorubicin, epirubicin, etoposide, idarubicinm, merbarone, mitoxantrone, novobiocin, pirarubicin, teniposide, valrubicin, and zorubicin); (iv) alkylating agents comprising nitrogen mustards (bendamustine, busulfan, chlorambucil, cyclophosphamide, estramustine phosphate, ifosamide, mechlorethamine, melphalan, prednimustine, trofosfamide, and uramustine), nitrosoureas (carmustine (BCNU), fotemustine, lomustine (CCNU), N-Nitroso-N-methylurea (MNU), nimustine, ranimustine semustine (MeCCNU), and streptozotocin), platinum-based (cisplatin, carboplatin, dicycloplatin, nedaplatin, oxaliplatin and satraplatin), aziridines (carboquone, thiotepa, mytomycin, diaziquone (AZQ), triaziquone and triethylenemelamine), alkyl sulfonates (busulfan, mannosulfan, and treosulfan), non-classical alkylating agents (hydrazines, procarbazine, triazenes, hexamethylmelamine, altretamine, mitobronitol, and pipobroman), tetrazines (dacarbazine, mitozolomide and temozolomide); (v) anthracyclines agents comprising doxorubicin and daunorubicin. Derivatives of these compounds include epirubicin and idarubicin; pirarubicin, aclarubicin, and mitoxantrone, bleomycins, mitomycin C, mitoxantrone, and actinomycin; (vi) enzyme inhibitors agents comprising FI inhibitor (Tipifarnib), CDK inhibitors (Abemaciclib, Alvocidib, Palbociclib, Ribociclib, and Seliciclib), PrI inhibitor (Bortezomib, Carfilzomib, and Ixazomib), PhI inhibitor (Anagrelide), IMPDI inhibitor (Tiazofurin), LI inhibitor (Masoprocol), PARP inhibitor (Niraparib, Olaparib, Rucaparib), HDAC inhibitor (Belinostat, Panobinostat, Romidepsin, Vorinostat), and PIKI inhibitor (Idelalisib); (vii) receptor antagonist agent comprising ERA receptor antagonist (Atrasentan), Retinoid X receptor antagonist (Bexarotene), Sex steroid receptor antagonist (Testolactone); (viii) ungrouped agent comprising Amsacrine, Trabectedin, Retinoids (Alitretinoin Tretinoin) Arsenic trioxide, Asparagine depleters (Asparaginase/Pegaspargase), Celecoxib, Demecolcine Elesclomol, Elsamitrucin, Etoglucid, Lonidamine, Lucanthone, Mitoguazone, Mitotane, Oblimersen, Omacetaxine mepesuccinate, and Eribulin.

Examples of immunotherapy include treatment with antibodies and antibody-drug conjugates including, but not limited to, alemtuzumab, Avastin (bevacizumab), Bexxar (tositumomab), CDP 870, and CEA-Scan (arcitumomab), denosumab, Erbitux (cetuximab), Herceptin (trastuzumab), Humira (adalimumab), IMC-IIF 8, LeukoScan (sulesomab), MabCampath (alemtuzumab), MabThera (Rituximab), matuzumab, Mylotarg (gemtuzumab oxogamicin), natalizumab, NeutroSpec (Technetium (99mTc) fanolesomab), panitumamab, Panorex (Edrecolomab), ProstaScint (Indium-Ill labeled Capromab Pendetide), Raptiva (efalizumab), Remicade (infliximab), ReoPro (abciximab), rituximab, Simulect (basiliximab), Synagis (palivizumab), TheraCIM hR3, tocilizumab, Tysabri (natalizumab), Verluma (nofetumomab), Xolair (omalizumab), Zenapax (dacliximab), Zevalin (ibritumomab tiuxetan (IDEC-Y2B8) conjugated to yttrium 90), Gilotrif (afatinib), Lynparza (olaparib), Perjeta (pertuzumab), Otdivo (nivolumab), Bosulif (bosutinib), Cabometyx (cabozantinib), trastuzumab-dkst (Ogivri), Sutent (sunitinib malate), Adcetris (brentuximab vedotin), Alecensa (alectinib), Calquence (acalabrutinib), Yescarta (ciloleucel), Verzenio (abemaciclib), Keytruda (pembrolizumab), Aliqopa (copanlisib), Nerlynx (neratinib), Imfinzi (durvalumab), Darzalex (daratumumab), Tecentriq (atezolizumab), and Tarceva (erlotinib).

“Checkpoint inhibitor therapy” is a form of cancer treatment that uses immune checkpoints which affect the way the immune system functions. Immune checkpoints can be stimulatory or inhibitory. Tumors can use these checkpoints to protect themselves from immune system attacks. Checkpoint therapy can block inhibitory checkpoints, restoring immune system function. Checkpoint proteins include programmed cell death 1 protein (PDCD1, PD-1; also known as CD279) and its ligand, PD-1 ligand 1 (PD-L1, CD274), cytotoxic T-lymphocyte-associated protein 4 (CTLA-4), A2AR (Adenosine A2A receptor), B7-H3 (or CD276), B7-H4 (or VTCN1), BTLA (B and T Lymphocyte Attenuator, or CD272), IDO (Indoleamine 2,3-dioxygenase), KIR (Killer-cell Immunoglobulin-like Receptor), LAG3 (Lymphocyte Activation Gene-3), TIM-3 (T-cell Immunoglobulin domain and Mucin domain 3), TIGIT (T-cell immunoglobulin and ITIM domain receptor) and VISTA (V-domain Ig suppressor of T cell activation).

Immunomodulatory drugs modify the response of the immune system by increasing (immunostimulators) or decreasing (immunosuppressives) the production of serum antibodies. Immunostimulators are prescribed to enhance the immune response against infectious diseases, tumors, primary or secondary immunodeficiency, and alterations in antibody transfer, among others. Immunosuppressive drugs are used to reduce the immune response against transplanted organs and to treat autoimmune diseases such as pemphigus, lupus, or allergies. Classes of immunostimulants include bacterial vaccines, colony stimulating factors, interferons, interleukins, therapeutic vaccines, vaccine combinations and viral vaccines. Other immunostimulants include glatiramer, plerixafor and elapegademase. Immunosuppressant drugs include corticosteroids, j anus kinase inhibitors, calcinerin inhibitors, mTOR inhibitors, IMDH inhibitors and biologic agents. Examples of immunosuppressant drugs include prednisolone, tofacitinib, cyclosporine, tacrolimus, sirolimus, everolimus, azathioprine, leflunomide, mycophenolate, abatacept, adalimumab, anakinra, certolizumab, etanercept, golimumab, infliximab, ixekizumab, natalizumab, rituximab, secukinumab, tocilizumab, ustekinumab, vedolizumab, basiliximab and daclizumab.

An immune metabolism modifying agent is an agent that changes the metabolism of immune cells.

A targeted therapy is a type of treatment that uses drugs or other substances to identify and attack specific types of cancer cells with less harm to normal cells. Some targeted therapies block the action of certain enzymes, proteins, or other molecules involved in the growth and spread of cancer cells. Other types of targeted therapies help the immune system kill cancer cells or deliver toxic substances directly to cancer cells and kill them. Targeted therapy may have fewer side effects than other types of cancer treatment. Most targeted therapies are either small molecule drugs or monoclonal antibodies. Targeted therapies include angiogenesis inhibitors, monoclonal antibodies, proteasome inhibitors and signal transduction inhibitors.

Anti-angiogenesis agents block the formation of new blood vessels that feed and nourish cancer cells. Examples of anti-angiogenesis agents include Axitinib, Bevacizumab, Cabozantinib, Everolimus, Lenalidomide, Pazopanib, Ramucirumab, Regorafenib, Sorafenib, Sunitinib, Thalidomide, Vandetanib and Ziv-aflibercept.

In an additional embodiment, the present invention provides a method of initiating, enhancing, or prolonging an anti-tumor response in a subject in need thereof by administering to the subject bryostatin-1 or a functional analog thereof, and an immunotherapeutic agent. In one aspect, the subject has a hematological cancer selected from a B-cell lymphoma, a T-cell malignancy, non-Hodgkin's lymphoma (NHL), mantle cell lymphoma (MCL), multiple myeloma (MM), acute myelogenous leukemia (AML), chronic myelogenous leukemia (CML), myelodysplastic syndrome, acute lymphoblastic leukemia (ALL), chronic lymphocytic leukemia (CLL), and hairy cell leukemia (HCL). In a further aspect, the immunotherapeutic agent is a monoclonal antibody, an antibody-drug conjugate, a chimeric antigen receptor (CAR)-T cell, chimeric antigen receptor natural killer (CAR-NK) cell, an anti-tumor vaccine or a combination thereof. In one aspect, immunotherapeutic agent is administered prior to, simultaneously with or following administration of bryostatin-1 or a functional analog thereof. In certain aspects, the initiating, enhancing or prolonging an anti-tumor response is an increase in CD5, CD19, CD 20, CD22, CD33, CD38, CD123, and/or BCMA expression following administration of bryostatin-1 or a functional analog thereof and the immunotherapeutic agent.

Initiating an anti-tumor response refers to the initiation of an immune response to a cancer cell. Enhancing an anti-tumor response refers to increasing the immune response to a cancer cell. Prolonging an anti-tumor response refers to making an immune response last longer.

In a further embodiment, the present invention provides a method of treating a cancer resistant to an immunotherapeutic agent in a subject by administering bryostatin-1 or a functional analog thereof and the immunotherapeutic agent to the subject. In one aspect, the cancer is a hematological cancer such as B-cell lymphomas, T-cell malignancies, non-Hodgkin's lymphoma (NHL), mantle cell lymphoma (MCL), multiple myeloma (MM), acute myelogenous leukemia (AML), chronic myelogenous leukemia (CIVIL), myelodysplastic syndrome, acute lymphoblastic leukemia (ALL), chronic lymphocytic leukemia (CLL) and hairy cell leukemia (HCL). In an additional aspect, the immunotherapeutic agent is a monoclonal antibody, a bispecific antibody, an antibody-drug conjugate, a chimeric antigen receptor (CAR)-T cell, a chimeric antigen receptor natural killer (CAR-NK) cell, an anti-tumor vaccine or a combination thereof. In certain aspects, the immunotherapeutic agent binds to or targets a protein selected from CD19, CD20, CD22, CD33, CD37, CD38, CD123 and BCMA. In various aspects, there is an increase in CD5, CD19, CD 20, CD22, CD33, CD38, CD123, and/or BCMA expression following administration of bryostatin-1 or a functional analog thereof and the immunotherapeutic agent.

The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLES

### Example 1

### Effect of Bryostatin on Cell Surface Protein Expression

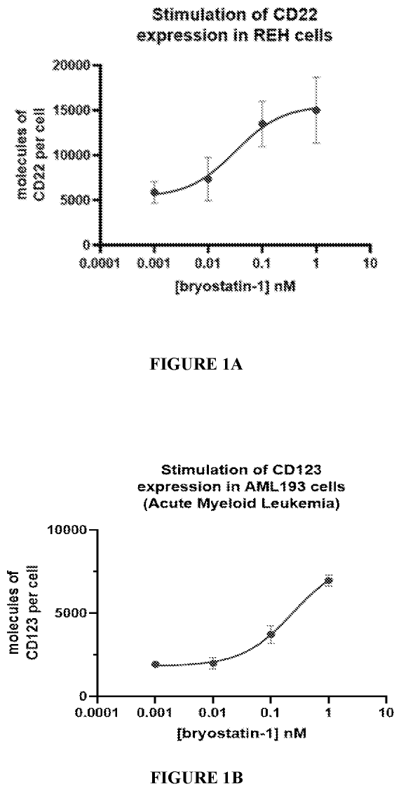

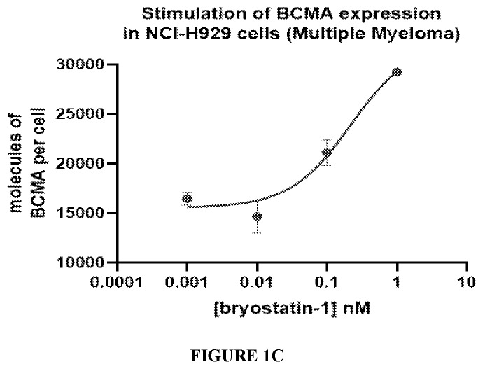

Bryostatin-1 has been examined in numerous tumor cell lines representing various hematological tumor types. Surface protein target expression was examined by flow cytometry with human cell lines stimulated with different concentrations of bryostatin-1. Human cell lines representative of different hematological disease indications of leukemia/lymphoma (Toledo, REH, DB), multiple myeloma (NCI-H929, U266B1, RPMI8226), and acute myeloid leukemia (Kasumi-1, AML-193, THP1) were used as target cell lines. Each cell line was treated with bryostatin-1 at 0.001, 0.01, 0.1, or 1 nM for either 24 hours or 72 hours in duplicate prior to measuring the surface molecule expression, quantitatively, of the following protein antigens by flow cytometry: CD19, CD20, CD22, CD33, CD37, CD38, CD123, CD269 (BCMA), and isotype control. Bryostatin-1 caused an upregulation of some antigens and not others. As shown in FIG. 1, CD22, CD123 and BCMA are all upregulated in response to bryostatin.

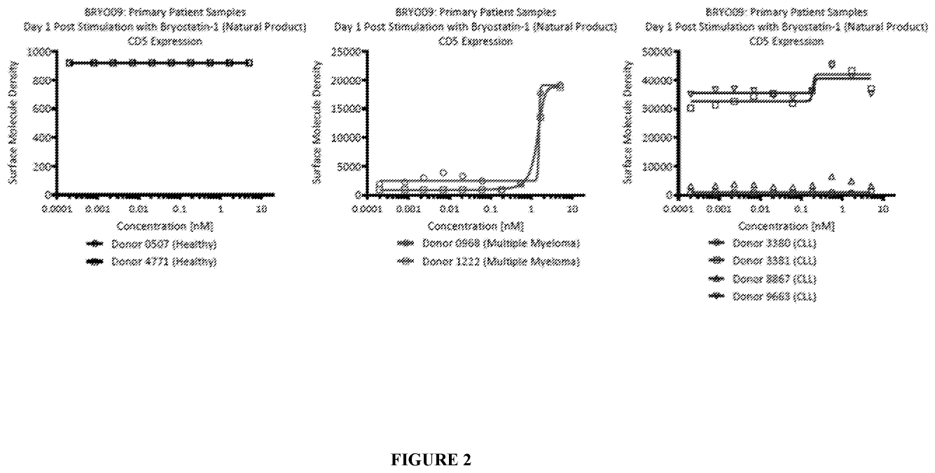

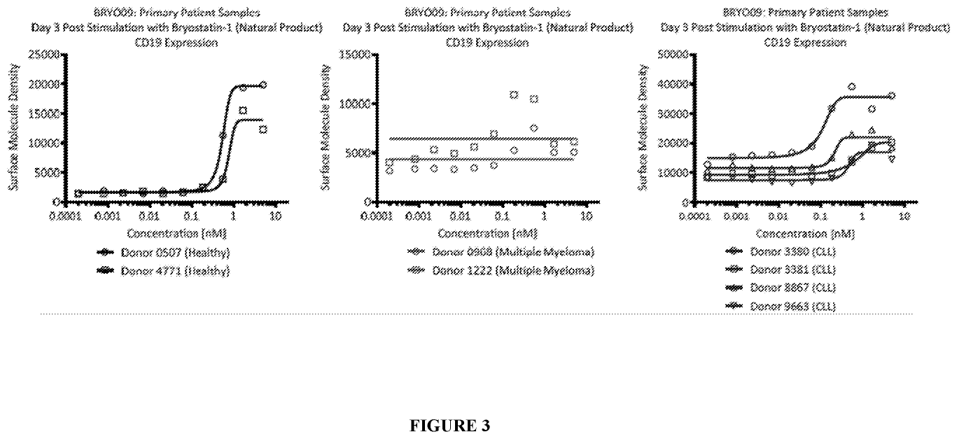

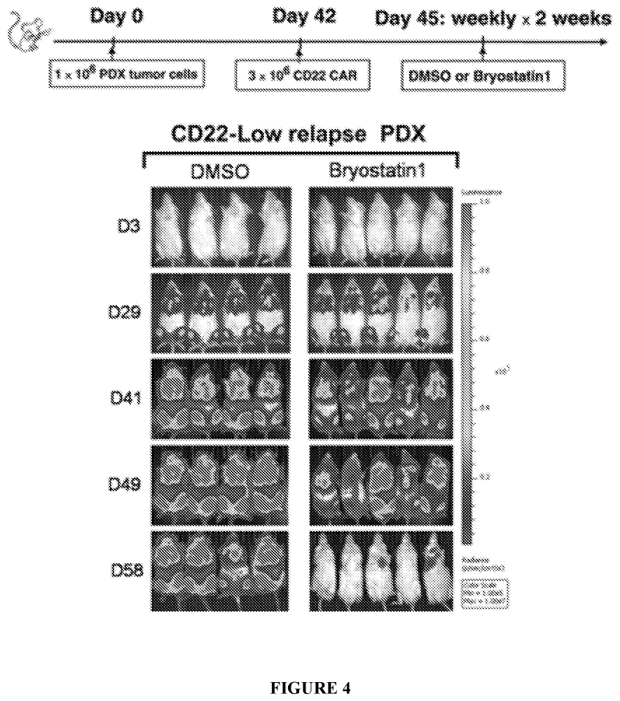

The ability of bryostatin-1 at multiple concentrations to modulate the surface expression of multiple antigens (CD5, CD19, CD20, CD22, CD37, CD200/OX2 and CD269/BCMA) at two time points on primary B cells from healthy donors (2 donors) or patients with multiple myeloma (2 patients, MM) or chronic lymphocytic leukemia (4 patients, CLL) was investigated. Examples of responses are shown in FIGS. 2-3. As can be seen, CD5 expression increases in response to bryostatin-1 after 24 h exposure in MM patients but not in healthy donors or CLL patients. In contrast, CD19 and CD22 are upregulated in healthy donors and CLL patients, but not MM patients.

### Example 2

### Effect of Bryostatin with Anti-CD22 Antibody on REH Cells

The ability of bryostatin-1 to enhance the effect of an anti-CD22 antibody, epratuzumab, modified in the experiment to mimic an antibody-drug conjugate was examined. The ability of epratuzumab to kill REH cells with and without bryostatin-1 was examined. Cells were inoculated on Day 0 and allowed to grow; on Day 42, CD22-directed CAR-T cells were administered and, as expected, the tumors did not respond. Administration of bryostatin-1 on Day 45 induced the tumors to respond, presumably by stimulating expression of CD22 in the relapsed tumor cells.

Bryostatin-1 lowered the EC50 for epratuzumab-induced killing of REH cells indicating an enhancement of epratuzumab potency in response to bryostatin-1.

| | EC₅₀ (μg/mL) |
|---|---|
| No Bryostatin-1 | 1.003 |
| 0.01 nM Bryostatin-1 | 0.814 |
| 1 nM Bryostatin-1 | 0.688 |
| 10 nM Bryostatin-1 | 0.584 |

Table 1: EC50 of responses of REH cells to epratuzumab with increasing concentrations of bryostatin-1.

Although the invention has been described with reference to the above examples, it will be understood that modifications and variations are encompassed within the spirit and scope of the invention. Accordingly, the invention is limited only by the following claims.

What is claimed is:

1. A method of treating cancer in a subject in need thereof comprising administering to the subject bryostatin-1 or a functional analog thereof and an immunotherapeutic agent, thereby treating the cancer.

2. The method of claim 1, wherein the cancer is a hematologic cancer selected from the group consisting of a B-cell lymphoma, a T-cell malignancy, non-Hodgkin's lymphoma (NHL), mantle cell lymphoma (MCL), multiple myeloma (MM), acute myelogenous leukemia (AML), chronic myelogenous leukemia (CML), myelodysplastic syndrome, acute lymphoblastic leukemia (ALL), chronic lymphocytic leukemia (CLL) and hairy cell leukemia (HCL).

3. The method of claim 1, wherein the immunotherapeutic agent is selected from the group consisting of a monoclonal antibody, a bispecific antibody, an antibody-drug conjugate (ADC), a chimeric antigen receptor (CAR)-T cell, a chimeric antigen receptor natural killer (CAR-NK) cell, an anti-tumor vaccine or a combination thereof.

4. The method of claim 3 wherein the immunotherapeutic agent is an anti-CD22 antibody.

5. The method of claim 3, wherein the CAR-T cell or the CAR-NK cell targets CD22.

6. The method of claim 3, wherein the ADC is inotuzumab ozogamicin.

7. The method of claim 1, further comprising administering to the subject an agent selected from the group consisting of an antibody directed against a (TAA), a chemotherapeutic agent, an ADC, a vaccine, an immunomodulatory drug, an immune metabolism modifying drug, a targeted therapy, radiation, an anti-angiogenesis agent, CAR-T therapy, CAR-NK therapy, an agent that reduces immune-suppression or a combination thereof.

8. The method of claim 1, wherein the immunotherapeutic agent is administered prior to, simultaneously with or following administration of bryostatin-1 or a functional analog thereof.

9. The method of claim 1, wherein the immunotherapeutic agent binds to or targets a protein selected from the group consisting of CD19, CD20, CD22, CD33, CD37, CD38, CD123 and BCMA.

10. The method of claim 1, wherein there is an increase in CD5, CD19, CD20, CD22, CD33, CD38, CD123 and/or BCMA expression following administration of bryostatin-1 or a functional analog thereof and the immunotherapeutic agent.

11. The method of claim 1, wherein the subject has CLL and there is an increase in CD19 and CD22 expression following administration of bryostatin-1 or a functional analog thereof and the immunotherapeutic agent.

12. The method of claim 1, wherein the subject has multiple myeloma and there is an increase in CD38 and/or BCMA expression following administration of bryostatin-1 or a functional analog thereof and the immunotherapeutic agent.

13. A method of initiating, enhancing, or prolonging an anti-tumor response in a subject in need thereof comprising administering to the subject bryostatin-1 or a functional analog thereof, and an immunotherapeutic agent, thereby initiating, enhancing, or prolonging an anti-tumor response in the subject.

14. The method of claim 13, wherein the subject has a hematological cancer selected from the group consisting of a B-cell lymphoma, a T-cell malignancy, non-Hodgkin's lymphoma (NHL), mantle cell lymphoma (MCL), multiple myeloma (MM), acute myelogenous leukemia (AML), chronic myelogenous leukemia (CML), myelodysplastic syndrome, acute lymphoblastic leukemia (ALL), chronic lymphocytic leukemia (CLL) and hairy cell leukemia (HCL).

15. The method of claim 13, wherein the immunotherapeutic agent is selected from the group consisting of a monoclonal antibody, a bispecific antibody, an antibody-drug conjugate (ADC), a chimeric antigen receptor (CAR)-T cell, a chimeric antigen receptor natural killer (CAR-NK) cell, an anti-tumor vaccine or a combination thereof.

16. The method of claim 13, wherein the immunotherapeutic agent is administered prior to, simultaneously with or following administration of bryostatin-1 or a functional analog thereof.

17. The method of claim 13, wherein the initiating, enhancing or prolonging an anti-tumor response is an increase in CD5, CD19, CD20, CD22, CD33, CD38, CD123 and/or BCMA expression following administration of bryostatin-1 or a functional analog thereof and the immunotherapeutic agent.

18. A method of treating a cancer resistant to an immunotherapeutic agent in a subject comprising administering bryostatin-1 or a functional analog thereof and the immunotherapeutic agent to the subject, thereby treating the cancer.

19. The method of claim 18, wherein the cancer is a hematological cancer selected from the group consisting of a B-cell lymphoma, a T-cell malignancy, non-Hodgkin's lymphoma (NHL), mantle cell lymphoma (MCL), multiple myeloma (MM), acute myelogenous leukemia (AML), chronic myelogenous leukemia (CML), myelodysplastic syndrome, acute lymphoblastic leukemia (ALL), chronic lymphocytic leukemia (CLL) and hairy cell leukemia (HCL).

20. The method of claim 18, wherein the immunotherapeutic agent is selected from the group consisting of a monoclonal antibody, an antibody-drug conjugate, a chimeric antigen receptor (CAR)-T cell, an anti-tumor vaccine or a combination thereof.

21. The method of claim 18, wherein the immunotherapeutic agent binds to or targets protein selected from the group consisting of CD19, CD20, CD22, CD33, CD37, CD38, CD123 and BCMA.

22. The method of claim 18, wherein there is an increase in CD5, CD19, CD20, CD22, CD33, CD38, CD123 and/or BCMA expression following administration of bryostatin-1 or a functional analog thereof and the immunotherapeutic agent.
